(19) United States
(12) Patent Application Publication
(10) Pub. No.: US 2015/0335257 A1
(43) Pub. Date: Nov. 26, 2015

(54) Hyperdrive and Neuroprobes for Stimulation Purposes

(71) Applicants: Bruce MCNAUGHTON, Leuven (BE); Gustaaf BORGHS, Leuven (BE); Arno AARTS, Leuven (BE); Peter PEUMANS, Leuven (BE)

(72) Inventors: Bruce MCNAUGHTON, Leuven (BE); Gustaaf BORGHS, Leuven (BE); Arno AARTS, Leuven (BE); Peter PEUMANS, Leuven (BE)

(73) Assignees: IMEC VZW, Leuven (BE); ATLAS NEUROENGINEERING BVBA, Leuven (BE); VIB, Gent (BE); Katholieke Universiteit Leuven, KU LEUVEN R&D, Leuven (BE)

(21) Appl. No.: 14/411,450

(22) PCT Filed: Jun. 28, 2012
(86) PCT No.: PCT/EP2012/062653

§ 371 Date: Dec. 26, 2014

(51) Int. Cl. A61B 5/04; A61N 1/05

(52) U.S. Cl. CPC A61B 5/04001; A61N 1/0551; A61B 2562/125

(57) ABSTRACT

A kit of parts for electrical stimulation and/or recording of activity of excitable cells in a tissue is described. The kit of parts comprises on the one hand a probe guiding means comprising a plurality of accommodation channels, each channel being adapted for accommodating a probe device having a plurality of stimulation means and/or recording means located on a die. At least one of the plurality of accommodation channels has a curved shape. The kit of parts also comprises at least one probe device for electrical stimulation and/or recording of activity of excitable cells in a tissue, the probe device comprising a plurality of stimulation means and/or recording means located on a die having a thinned and etched surface for providing flexibility to the probe device.

### FIELD OF THE INVENTION

The invention relates to the field of brain research or stimulation instrumentation. More particularly, the present invention relates to a method and system for recording an electrical field generated by or applying an electrical field to cells.

### BACKGROUND OF THE INVENTION

Brain research instrumentation for in-vivo recording of an electrical field generated by neural cells, e.g. for observing an extracellular field potential present in nervous tissue, and/or for applying an electrical field in-vivo to neural cells, may be useful for neurophysiological and/or neuropharmacological studies in animals or humans. Such instrumentation may also find clinical application such as for diagnostic purposes, e.g. for monitoring, or for therapeutic purposes, e.g. for neurostimulation.

Several methods for in-vivo recording of electrical fields generated by neural cells and/or in-vivo application of electrical fields to neural cells are known in the art. A known system for brain research or stimulation may, for example, comprise at least one electrode for implantation at a particular central nervous system (CNS) region, such that the electrical field generated by neurons in close proximity to the tip of the at least one electrode may be characterized, e.g. measured, or influenced, e.g. by exciting or inhibiting the neurons through application of an electrical field. As the number of electrodes increases, e.g. by providing electrodes at multiple CNS regions, the information content from the obtained data also increases, as relationships between firing sequences in different regions may reveal detailed information about neural connectivity and functional relationships between these regions. When the redundance of information obtained from different electrodes reaches a minimum, the amount of information which may be obtained can be proportional to the square of the number of electrodes, e.g. proportional to the number of electrode pair combinations.

In order to accommodate a large number of electrodes, e.g. multichannel electrodes, a system is disclosed in U.S. Pat. No. 5,928,143 which provides an implantable microdrive array. In this disclosure, a plurality of multichannel electrodes are inserted in a positioning array which is small and lightweight such that the device may be carried on the skull of an animal subject while freely moving and awake. The positioning array comprises a plurality of elongate guide cannulae with lower ends arranged in parallel and upper ends which are inclined outwardly. The recording electrodes are slidably carried within each of the guide cannulae, such that the positions of the electrodes are independently adjustable. By moving each electrode to a suitable position the data acquisition may thus be optimized.

However, in addition to providing a plurality of electrodes at different regions of the brain, providing a plurality of electrodes in close proximity to each other may also be advantageous, even though this implies a large signal redundancy. It is known that cells with different ratios of distances from two electrode tips have different spike-amplitude ratios when recorded on two channels. This principle gave rise to electrode designs in which electrical potentials from single neurons or small clusters of neurons may be isolated by using several electrodes in a fixed position in space relative to each other, such as in the stereotrode and tetrode electrode arrangement. For example, a tetrode may comprise a bundle of four thin electrode wires, e.g. four wires of 30 μm in diameter. These wires are in close proximity to each other such that the electrical fields observed by each electrode are generated by substantially overlapping neuron populations, but the exact waveform of the electrical field contribution of individual neurons differs on each wire. For example, the four wires may be embedded in a rod composed of an electrically insulating material, e.g. quartz glass, such that the wires end in contact zones at the surface of such rod. Such a rod may be cylindrical with a pointed end on which the contact zones are arranged, e.g. such that the centers of these contact pads correspond to the vertices of a regular tetrahedron.

In European Patent Application No. EP 1 723 983, a probe device is disclosed which comprises a substrate having a die on top thereof. The die comprises a plurality of stimulation and/or recording sites, e.g. contact pads, for example tens to hundreds of such sites. The substrate is furthermore folded into a cylindrical or conical shape. The probe thus formed may be used to acquire a spatial distribution at a high resolution of an in-vivo electric field in neural tissue, such that the firing of individual neurons or small neuron clusters in the vicinity of the probe may be observed accurately, even when the individual channels provide noisy signals.

### SUMMARY OF THE INVENTION

It is an object of embodiments of the present invention to provide good methods and systems for probing electrical fields.

It is an advantage of embodiments according to the present invention that the probes are adapted for use with a hyperdrive, the hyperdrive being a system for introducing a plurality of probes into a human or animal body.

It is an advantage of embodiments according to the present invention that highly flexible probes are provided that can be introduced in a hyperdrive without having a substantial risk of breaking.

It is an advantage according to embodiments of the present invention that a substantially higher density of recording sites can be obtained compared to the use of a neuroprobe as such or the use of a hyperdrive with tetrodes.

It is an advantage of embodiments according to the present invention that systems and methods are provided allowing control of the depth of the probes individually.

The above objective is accomplished by a method and device according to the present invention.

The present invention relates to a kit of parts for electrical stimulation and/or recording of activity of excitable cells in a tissue, the kit of parts comprising
a probe guiding means comprising a plurality of accommodation channels, each channel being adapted for accommodating a probe device having a plurality of stimulation means and/or recording means located on a die, wherein at least one of the plurality of accommodation channels has a curved shape,
at least one probe device for electrical stimulation and/or recording of activity of excitable cells in a tissue, the probe device comprising a plurality of stimulation means and/or recording means located on a die having a thinned and etched surface for providing flexibility to the probe device. It is an advantage of embodiments according to the present invention that the thinned and etched die results in a flexibility allowing the probe to be insertable into the accommodation channel with curved shape without breaking. A 4 cm tall probe can for example be bendable over 180°.

At least one probe device may have a die with a length of at least 30 mm. It is an advantage of embodiments according to the present invention that the probes can be sufficiently long so that they can be inserted in a human or animal body through the hyperdrive, without the need for fitting the connection between the die of the probe and the measurement apparatus in the probe guiding means. Whereas in embodiments according to the present invention reference is made to a probe guiding means, reference also may be made to a hyperdrive.

The probe guiding means may comprise a probe positioning means adapted for individually controlling a position for probe devices in different accommodation channels. It is an advantage of embodiments according to the present invention that a different positioning of different probe devices used with the same probe guiding means can be performed, allowing for accurate positioning of different probe devices while also reaching a high density.

The accommodation channels of the probe guiding means may have at least one end where the accommodation channels are adjacent and/or the accommodation channels of the probe guiding means have at least one end where the different accommodation channels are spaced from each other. It is an advantage of embodiments according to the present invention that a high density of measurement or stimulation sites can be obtained, by inserting a plurality of probe devices closely adjacent one another in the tissue. It is an advantage of embodiments of the present invention that sufficiently space can be provided so as to allow the use of a connection means to each of the probe devices for connecting the probe device with a measurement system.

The kit of parts may be adapted for simultaneously recording or stimulating at at least 1500 sites. It is an advantage of embodiments according to the present invention that a high number of measurement channels can be established using the kit of parts. In some embodiments a density of 256 measurement channels per 0.5 mm³ or higher can be obtained.

The present invention also relates to a probe device for electrical stimulation and/or recording of activity of excitable cells in a tissue, the probe device comprising a plurality of stimulation means and/or recording means located on a die, the die having a thinned and etched surface for providing flexibility to the probe device.

The die may have a length of at least 30 mm. According to some embodiments of the present invention, the length of the die is at least 40 mm.

The present invention furthermore relates to a method for manufacturing a probe device as described above, the method comprising processing a number of stimulation sites and/or recording sites in an elongated die, thinning the die to a thickness below 50 μm, and applying an etching or dry polishing process for removing sub-surface damage induced by the processing or thinning for increasing the flexibility of the die.

Applying an etching process for removing sub-surface damage may comprise performing any of a dry etch or a wet etch.

Applying a dry etch may comprise applying an SF₆ based isotropic plasma etch. Applying a wet etch may comprise applying an etch in TMAH.

The present invention also relates to a probe guiding means for use in electrical stimulation and/or recording of activity of excitable cells in a tissue, the probe guiding means comprising a plurality of accommodation channels, each channel being adapted for accommodating a probe device having a plurality of stimulation means and/or recording means located on a die, wherein at least one of the plurality of accommodation channels has a curved shape.

The probe guiding means may comprise a probe positioning means adapted for individually controlling a position for probe devices in different accommodation channels.

The accommodation channels of the probe guiding means may have at least one end where the accommodation channels are adjacent. The accommodation channels of the probe guiding means may have at least one end where the different accommodation channels are spaced from each other.

The present invention also relates to a method for determining a pattern of signals from excitable cells in a tissue, using a kit of parts as described above, the method comprising inserting a plurality of probe devices in the probe guiding means, and recording electrical activity of excited cells.

The present invention furthermore relates to a device for determining a pattern from excitable cells in a tissue by means of a kit of parts as described above, the device comprising a kit of parts according to any of claims 1 to 5 for recording electrical activity of excited cells and generating corresponding activity signals, and processing means for comparing the generated activity signals with pre-determined activity signals for the excited cells.

Particular and preferred aspects of the invention are set out in the accompanying independent and dependent claims. Features from the dependent claims may be combined with features of the independent claims and with features of other dependent claims as appropriate and not merely as explicitly set out in the claims.

These and other aspects of the invention will be apparent from and elucidated with reference to the embodiment(s) described hereinafter.

### BRIEF DESCRIPTION OF THE DRAWINGS

FIG. 1a and FIG. 1b illustrate highly flexible neuroprobes according to an embodiment of the present invention.

FIG. 2a to FIG. 2d illustrate different views of a probe guiding means for accommodating a plurality of neuroprobes, according to an embodiment of the present invention.

FIG. 3a and FIG. 3b illustrate a kit of parts showing a hyperdrive with inserted neuroprobes, according to an embodiment of the present invention.

FIG. 4 illustrates a method for manufacturing a neuroprobe having a good flexibility, according to an embodiment of the present invention.

FIG. 5 illustrates an x-ray image of a system comprising a hyperdrive and a set of neuroprobes inserted in the hyperdrive, illustrating the use of a kit of parts as described above, according to an embodiment of the present invention.

FIG. 6 illustrates an example of the insertion of a flexible neuroprobe into a channel for the hyperdrive, illustrating features of embodiments of the present invention.

FIG. 7 is a photograph of a hyperdrive according to embodiments of the present invention.

The drawings are only schematic and are non-limiting. In the drawings, the size of some of the elements may be exaggerated and not drawn on scale for illustrative purposes.

Any reference signs in the claims shall not be construed as limiting the scope.

In the different drawings, the same reference signs refer to the same or analogous elements.

### DETAILED DESCRIPTION OF ILLUSTRATIVE EMBODIMENTS

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Moreover, the terms top, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Whereas methods and systems are described with reference ton neuro-probe devices, embodiments of the present invention are not limited thereto and can also relate to other probe devices, such as probe devices for implantation in muscular tissue or in cardiac tissue for stimulating excitable cells within these tissues.

Where in embodiments according to the present invention reference is made to excitable cells in tissues, reference is made to cells that can be modulated or excited by electric fields, in that way providing a possible therapeutic approach or allowing research for several disorders affecting these tissues. Such cells may be situated in nervous tissue, cardiac tissue, muscular tissue, etc.

In a first aspect, the present invention relates to a kit of parts for electrical stimulation and/or recording of activity of excitable cells in a tissue. The kit of parts may be especially suitable for neuroprobing, although embodiments of the present invention are not limited thereto. The kit of parts comprises both a probe guiding means allowing guiding of different probe devices towards an accurate position for probing, and at least one die-based probe device comprising a plurality of recording or stimulation means located on the die. The probe guiding means according to embodiments of the present invention comprises a plurality of accommodation channels, each channel being adapted for accommodating a die-based probe device. At least one of the plurality of the accommodation channels thereby has a curved shape. The at least one probe device comprises a plurality of stimulation means and/or recording means located on a die. According to embodiments of the present invention, the die thereby has a thinned and etched surface. The etched surface thereby renders the probe device sufficiently flexible for use with the probe guiding means, i.e. for being accommodated in the accommodation channel having a curved shape.

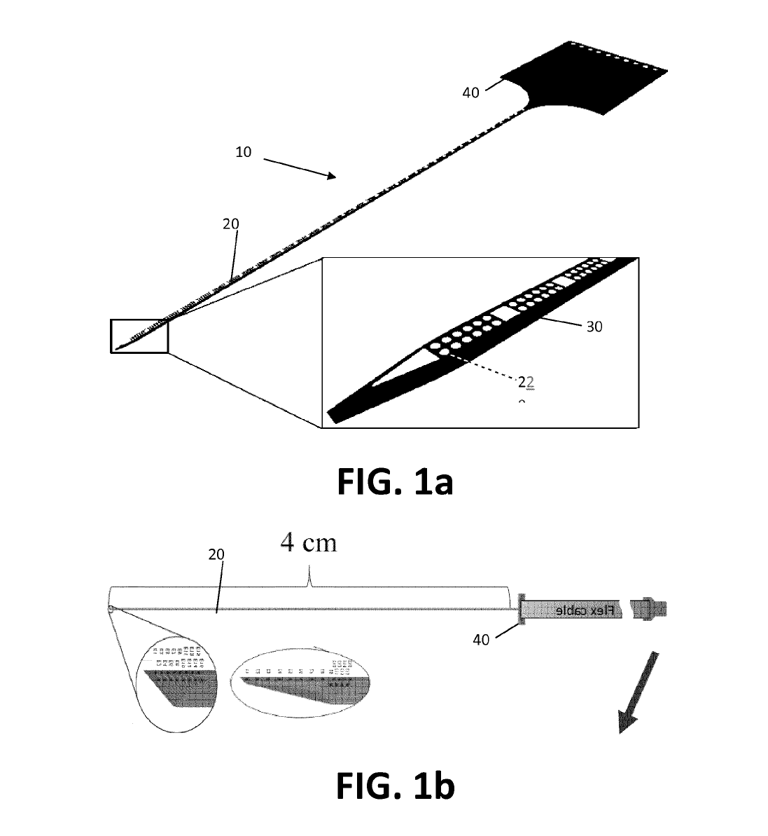

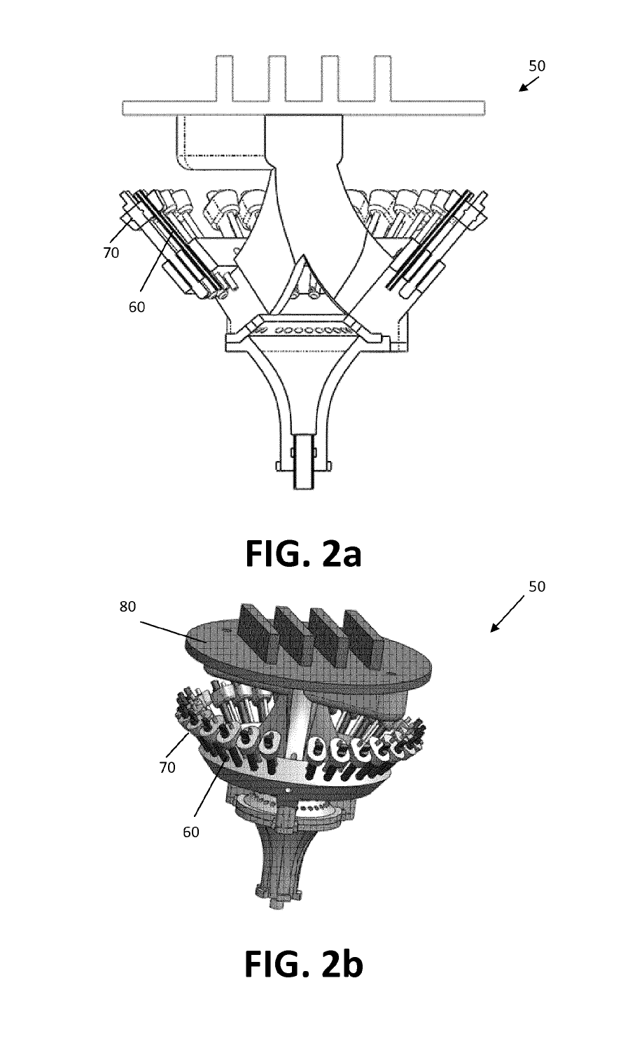

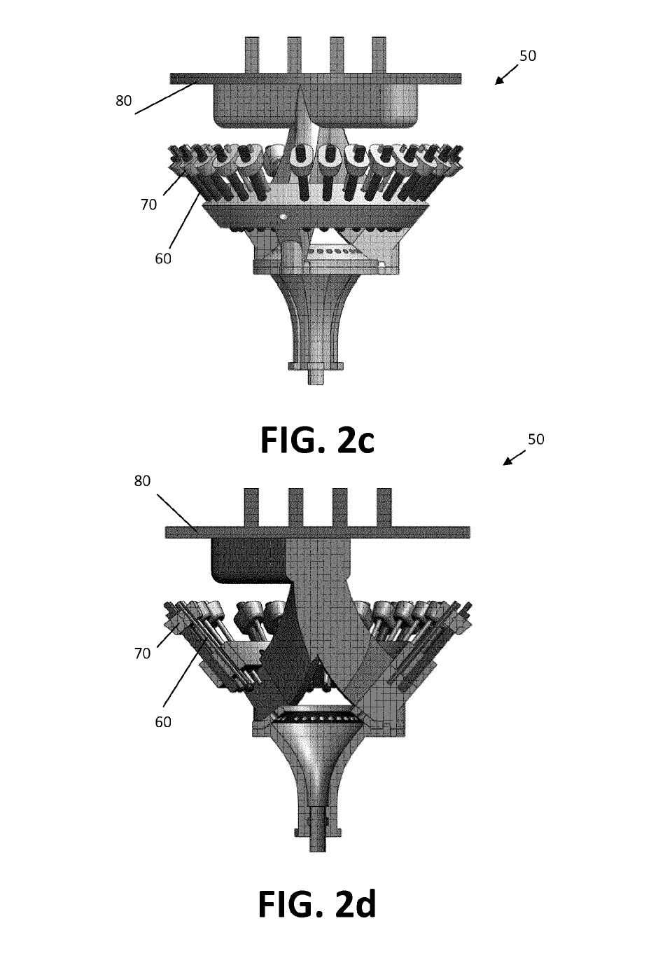

By way of illustration, embodiments of the present invention not being limited thereto, further features and advantages of at least some embodiments are described with reference to the exemplary kit of parts, illustrated in FIG. 3 and with reference to the probe device and the probe guiding means illustrated in FIG. 1 and FIG. 2 respectively.

The exemplary kit of parts comprises on the one hand a probe device. The probe device comprises a plurality of stimulating means and/or recording means. Each of the stimulating means or the recording means may correspond with a stimulation or recording site. Each of such a site may be adapted for recording or applying a signal to a particular position in the tissue. The latter can for example be arranged in an array, although embodiments of the present invention are not limited thereto. A stimulation means may for example be a stimulation transducer or micro-electrode for stimulating a part of the tissue. A recording means may for example be a recording transducer or micro-electrode for measuring activity of a part of the tissue. The recording electrode may be an active device, e.g. transistor, generating local signal amplification and high to low impedance conversion.

In some embodiments, both a plurality of stimulation means and a plurality of corresponding recording means may be present.

One example of a stimulation means may be a microelectrode which may comprise a noble metal (e.g. Au, Pt, Ir or IrOx). Preferably, Pt and/or Ir may be used for the delivery of the stimulation pulses when in contact with excitable cells, e.g. neurons. The microelectrodes should be able to deliver monophasic cathodic or biphasic pulses generated by a voltage controlled pulse generator (0 to 20 V stimulus amplitude, 20 to 1000 sec, for example between 60 and 200 sec pulse duration and 2 to 1000 Hz, for example between 60 and 200 Hz frequency). Field-effect transistors (FETs) may, for example, be used as recording transducers or micro-electrodes for recording of the cell activity. A typical size of the surface of the stimulating means or recording means by which the tissue can be contacted may be a width and length between 5 μm and 100 μm, preferably between 5 μm and 50 μm, more preferably between 5 μm and 30 μm and most preferably between 5 μm and 10 μm. The size of the stimulation and/or recording means determines the resolution of the probe device. Therefore, in order to obtain a good resolution, each stimulation means and/or recording means may preferably be as small as possible because the better the resolution is, the more precise the controllability of the probe device becomes. However, the smaller the surface area of the stimulation electrode, the higher the charge density will become (Coul/cm<2>). The charge density determines the amount of current that can be delivered, and this must happen without damaging the tissue where the probe device is positioned. Advantageously, the number of stimulation and/or recording means may be at least 5, more advantageously at least 10 sites. The number of stimulation and/or recording means that may be present on the die, may e.g. be 16, 32, 64 or any other suitable number.

According to embodiments of the present invention, the stimulation and/or recording means are processed on a die, typically being an elongated die. The die for example may be a silicon die, although e.g. other semiconductor materials such as e.g. GaAs, can also be used. The stimulation means and/or recording means may be implemented on the die using micro-fabrication techniques known by persons skilled in the art, such as for example, IC or CMOS standard and non standard processes. According to embodiments of the present invention, the die is a thinned die, whereby thinning e.g. may be performed using any suitable method, such as e.g. mechanical or chemical polishing or by a combination of both. According to embodiments of the present invention, the surface to which thinning is applied furthermore is an etched surface. One or more of a dry etch, wet etch or a dry polishing technique is performed for removing sub-surface damage introduced in the surface by the thinning effect and diminishes the stress relief significantly. Rough grinding techniques typically induce surface damage, which can propagate up to several tens of microns into the surface. Fine grinding is used to reduce those surface defects, however, there is still subsurface present.

As described above, the die typically may have an elongated shape. The length of the probe device may be at least 30 mm, e.g. more than 40 mm. Such a length does not only provide the possibility to use it with a probe guiding means as part of the kit of parts, but also allows for deep brain stimulation, one of the possible applications of embodiments of the present invention. The width of the die or more specific, the probe, can be 200 um, 150 um, 100 um or even down to a few microns wide. The base of such probe can be wider due to interconnect reasons. Such probe fits perfectly in the guiding tube of the hyperdrive.

The die furthermore may comprise contacts for contacting the stimulation and/or recording sites. Such contacts typically may be made of Al or Au or any other suitable noble metal. The contacts may be covered with a biocompatible insulating coating such as for oxides (e.g. IrOx, Ta2O5, SiO2, ZrO2), Si3N4, polymers (e.g. parylene C, parylene N, silicone rubbers, polyimide) or biocompatible epoxies. This biocompatible insulating coating provides the advantageous passivation.

The die may be bonded to a biocompatible substrate, also called packaging substrate, with a given geometry suitable for implantation in the relevant anatomic target. A 3D field distribution may therefore be implemented and the geometry of the probe device should enable this. Therefore, ideally, the probe device, and therefore the substrate thereof, may have a substantially cylindrical shape or may have a conical cross-section with the active pixels or stimulation/recording means distributed on the external site and thus in contact with the tissue, e.g. the brain tissue. Due to the stimulation transducers and recording means being distributed and bonded onto a substrate having a substantially cylindrical shape or a shape with a conical cross-section, the electrical field distribution can be controlled. Furthermore, recordings of electrical activity of excitable cells can be performed in three dimensions. The probe device according to embodiments of the present invention may be implemented such that it has no sharp edges and in that way damage to the tissue is avoided.

The probe device may further be equipped with or be adapted for cooperating with signal processing and control circuitry. The signal provided by the recording transducers can be processed by a controller, e.g. a micro-processor unit. Applying signals to the stimulation means may for example also be performed by a controller, e.g. a micro-processor unit. By means of steering electronics, specific stimulation and/or recording means can be controlled allowing to control the stimulation pattern, and for example also to reconfigure the stimulation pattern, e.g. if the probe device would have moved. The steering electronics can be completely external to the tissue or may be distributed between the probe device and an external part thereof. Although the term ‘external’ is used, this does not mean that the steering electronics are necessarily outside the body of the patient. For example, this includes that the steering electronics may be implanted not in the brain itself, but e.g. below the skin.

According to some embodiments, the at least one probe device furthermore may comprise a connection means for connecting the probe device to an external controlling or measuring unit. Such a connection means may for example be flexible cable bonded to the die or the substrate on one side and suitable for connecting to a connector input at the other side. One example of a connector that could be used is a ZIF connection, although embodiments of the present invention are not limited thereto.

An example of a probe device according to embodiments of the present invention is shown in FIG. 1a and FIG. 1b. Both illustrate flexible probe devices comprising a plurality of stimulating and/or recording means. In FIG. 1a and FIG. 1b, a probe device 10 with an elongated die 20 comprising a plurality of stimulating and/or recording means 22 is shown. Furthermore, the flexible substrate 30 and a connection means 40 for connecting to a controller, also is shown.

According to embodiments of the present invention, the kit of parts also comprises a probe guiding means, adapted for accommodating a plurality of probe devices. The probe guiding means, also referred to as hyperdrive, may be made of any suitable material. It may be made of a casted or molded material. The probe guiding means according to embodiments of the present invention comprises a plurality of accommodation channels. Each of these channels is adapted for accommodating a die-based probe device as described above. The accommodation channels may be arranged such that they allow to insert different die-based probe-devices closely next to each other in the tissue, thus resulting in a high density of stimulating means and/or recording means in the tissue. The accommodation channels therefore may at one side being positioned adjacent next to each other. In the present example, at the other side, the accommodation channels are not positioned adjacent each other, but are more widely spaced. The latter can assist in providing sufficient space for easily connecting and/or inserting the probe devices. On the other hand, this also implies that at least one of the channels has a curved shape. In view of the curved shape and in order to allow inserting the probe device into such a curved accommodation channel, as described above, the probe devices have an etched surface, whereby through etching sub-surface damage from the thinning has been removed, resulting in a thin and flexible probe device, insertable without breaking into the accommodation channel. The number of accommodation channels that can be provided in the probe guiding means may be any suitable number, e.g. 2 or more, more than 4, more than 8, at least 16, etc.

According to embodiments of the present invention, the probe guiding means may comprise a probe positioning means adapted for individually determining the position of die-based probe devices in the tissue. Such a probe positioning means thus may provide the possibility for controlling the depth of the probes individually. The probe positioning means may be a mechanical means, electronic means, electromechanical means, etc. An example of a probe guiding means according to embodiments of the present invention is shown in FIG. 2a to FIG. 2d illustrating different side views of a probe guiding means. A probe guiding means 50 comprising a plurality of accommodation channels 60 is shown, whereby the accommodation channels 60 are adjacent at one end of the accommodation channels and spaced from each other at the other end. It can be seen that at least one accommodation channel is curved. Furthermore, a probe positioning means 70 also is shown, as well as a further connection system 80.

FIG. 3a and FIG. 3b illustrate a probe device 10 inserted in a probe guiding means 50 comprising a plurality of accommodation channels and a further connector system 80. It can be seen that the probe device 10 should be highly flexible in order to be able to insert it in the hyperdrive without breaking. The die portion of the probe thus is bent in the accommodation channels.

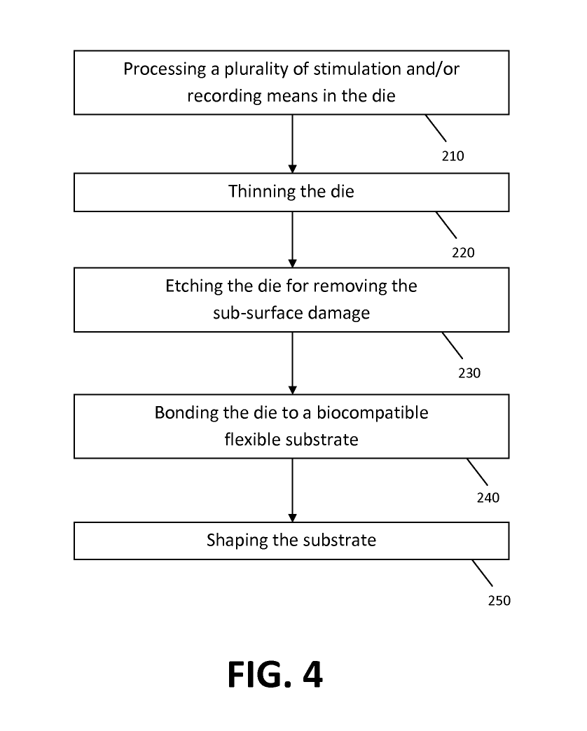

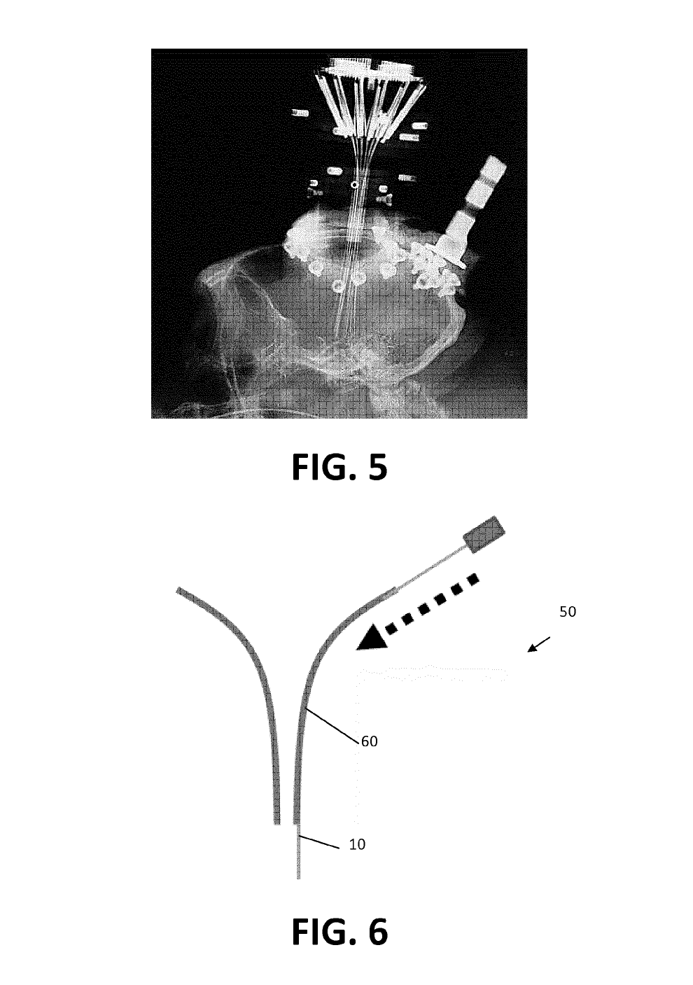

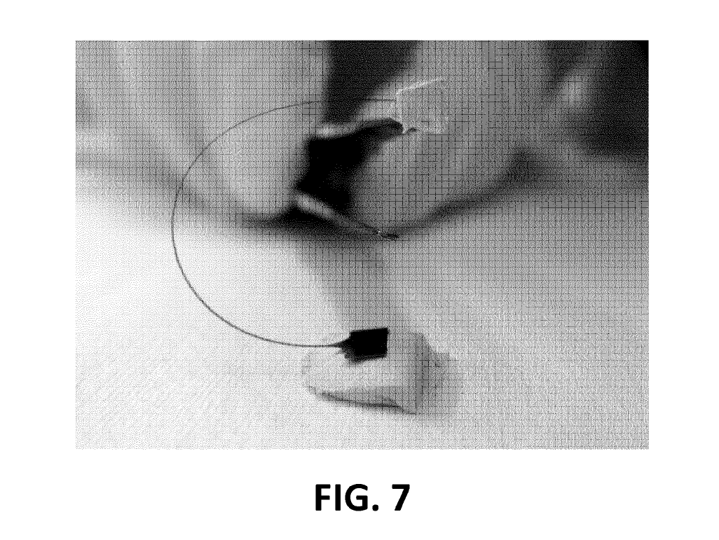

By way of illustration, embodiments of the present invention not being limited thereto, an example of an x-ray image illustrating the system in position during use is shown in FIG. 5. FIG. 6 illustrates the insertion of the probe device in an accommodation channel of the probe guiding means. In FIG. 7, a photograph of a probe device and the ability to bend is illustrated.

In one aspect, embodiments according to the present invention also relate to a probe device for electrical stimulation and/or recording of activity of excitable cells in a tissue, as described in the first aspect. Such a probe device may have the same features and advantages as described in the first aspect.

In another aspect, embodiments according to the present invention further relate to a probe guiding means for accommodating die-based probe-devices and guiding them into the tissue to be studied. Such probe guiding means may have the same features and advantages as the probe guiding means described in the first aspect of the present invention.

In a further aspect, the present invention relates to a method for manufacturing a probe device as described above. The method for manufacturing is particularly suitable for creating probe devices having sufficient length and sufficient flexibility for co-operating with a probe guiding means for guiding die-based probe devices. According to embodiments of the present invention, the method comprises processing a number of stimulation sites and/or recording sites in an elongated die, thinning the die to a thickness below 50 μm, and applying an etching or polishing process for removing sub-surface damage induced by the processing or thinning for increasing the flexibility of the die. The etching process for removing sub-surface damage may comprise performing at least one of a dry etch, a wet etch or a dry polishing technique. One example of a dry etch that may be applied is a fluorine based plasma or chlorine based plasma etch. An example of a wet etch that may be applied can be TMAH (Tetramethylammonium hydroxide) based or EDP (aqueous solution of ethylene diamine and pyrocatechol) base or HF based.

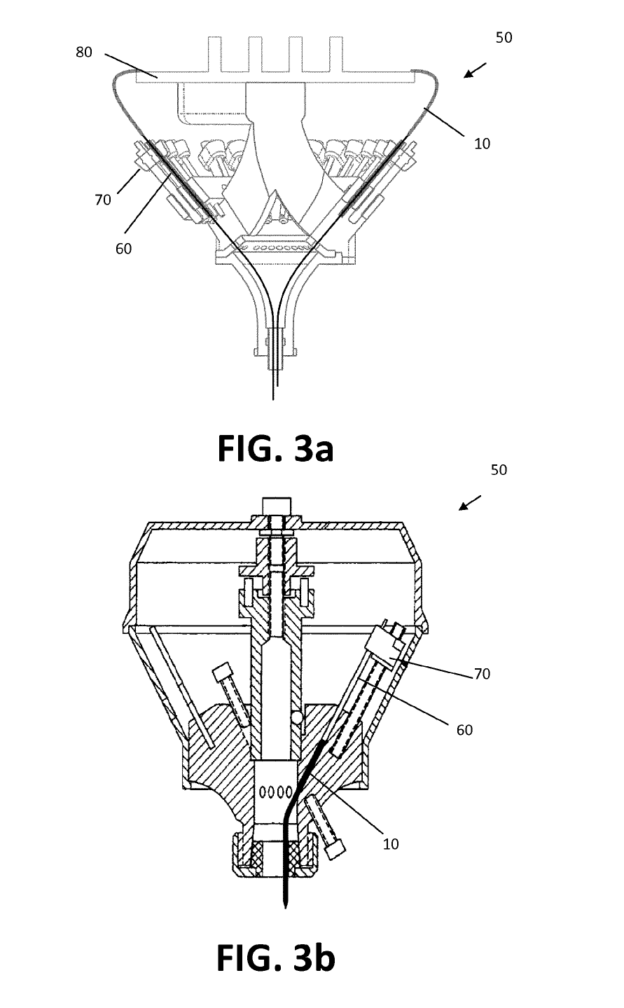

Referring to FIG. 4, an exemplary method 200 for manufacturing a probe device according to embodiments of the first aspect of the present invention is shown. This method comprises the step of processing 210 a number of stimulation means and/or recording sites at one end of an elongated die. First, a die may be obtained. Such a die may have an elongated shape or may be processed at the start of the method or later in the method to have an elongated shape. A number of stimulation means and/or recording means may be formed on an elongated die, e.g. a silicon die, or alternatively on another semiconductor die material, such as GaAs or silicon on insulator (SOI). This processing 210 may be performed by micro-fabrication techniques as commonly known by a person skilled in the art, such as standard or non-standard CMOS or IC processes. The elongated die may have a thickness of between 300 μm and 1 mm, such as 850 μm. Thus, the array of stimulation sites and/or recording sites may be applied on a standard die by a standard process. Contacts for the stimulation sites and/or recording sites may, for example, be provided using standard CMOS metallization processes. Such contacts may be composed of electrically conductive materials, for example aluminum, gold, platinum or other suitable conductive metals, alloys or composite materials.

In a second step, the method 200 comprises thinning 220 the die to a thickness below 50 μm, preferably below 25 μm, more preferably down to 10 μm, and most preferably down to 5 μm. This thinning 220 may be achieved by standard CMOS processing on semiconductor substrates, e.g. Si, GaAs or SOI. This thinning down is performed to reduce the thickness of the die such that the die becomes flexible. Thinning 220 may comprise a bulk semiconductor thinning technique, e.g. mechanical or chemical polishing or a combination of both.

In a third step, the method 200 comprises applying 230 an etching process for removing sub-surface damage induced by the processing 210 or thinning 220 for increasing the flexibility of the die. Such sub-surface damage may typically comprise disturbances of the bulk crystal lattice by the processing 210 and/or thinning 220 steps, for example by grinding or chemical polishing. Such sub-surface damages may be of increasing concern as the die is reduced to a decreasing thickness. Not only can damages extend from the backside to the frontside of the thin wafer and impair functionality of the device, but the damages can also induce stress into the substrate. Sub-surface damage can mechanically weaken the thin die, thus making it prone to breakage. Two types of damage may be of particular concern: micro-cracks and dislocations. Point-defects on the other hand may be grown into the lattice during production, but are typically not introduced by thinning, unless maybe close to the surface by RIE. However, micro-cracks, e.g. planar defects, may at one end of the plane give rise to huge stress concentrations.

The thinning 220 may be performed to a thickness above the final intended thickness, e.g. to take into account a further reduction of the thickness of the die by the step of applying 230 an etching process for removing sub-surface damage. This applying 230 of an etching process may comprise at least one etching step, for example an incremental etching where in each etching step a thin layer is removed, for example reducing the thickness by 5 to 10 μm. Applying 230 an etching process may thus comprise a dry etching, e.g. the application of an SF₆-based isotropic plasma etch. Alternatively or additionally, the application 230 of an etching process may comprise a wet etching, e.g. using an anisotropic semiconductor etchant, e.g. an anisotropic silicon etchant such as 25% TMAH at 80° C.

In a further step 240, the thinned die can be bonded to a substrate. The substrate may be a biocompatible flexible substrate that can subsequently be folded to acquire a desired shape, preferably a tubular or conical shape. The substrate may, for example, comprise biocompatible material such as any of parylene C, parylene N, polyimide, polysiloxane rubber or teflon, but may also comprise a noble metal (e.g. Au, Pt, Ir), titanium, oxides (e.g. IrOx, Ta2O5, SiO2, ZrO2), Si3N4 or biocompatible epoxies. The material the substrate is formed of should be such that cytotoxicity and material degradation is prevented when the probe device is implanted in the tissue. Typically, the die comprising the stimulation/recording means forms the active part of the probe device. The substrate represents a probe shaft and it may be used to anchor the probe device. The packaging or bonding method may be based on either wire bonding or flip chip assembly. Wire bonding or flip chip assembly both are known techniques in the field of manufacturing probe devices and therefore are not further described in detail here.

In a next step 250, the substrate may further be shaped, e.g. folded or bent. The structure may fold or bend by itself due to internal stress in the sheet, i.e. a sort of curling, or a lamination method could be used. Once folded, the sides of the probe device can be attached to each other, so as to form a substantially cylindrical shape or a shape having a conical cross-section. This may be done by any suitable attaching means, such as gluing of the sides onto each other, which may be thermally induced (local heating) or may be performed by using biocompatible glues such as e.g. UV curable epoxies or silicones. Alternatively also a flat probe device surface can be used or maintained and the sides of the die need not to be attached to each other.

In a further aspect, the present invention also relates to a method for determining a stimulation pattern for application to excitable cells in a tissue. According to embodiments of the present invention, the method comprises using a kit of parts as described above thereby inserting a plurality of probe devices in the probe guiding means, into the tissue to be stimulated and/or from which a signal is to be recorded. The method also comprises recording signals and optionally comparing recorded electrical activity to predetermined activity values of said excited cells. The latter may allow obtaining information regarding the excited cells and the tissue under study. In another aspect, the present invention also relates to a corresponding device.

1-15. (canceled)

16. A method for manufacturing a probe device, comprising:
processing a number of stimulation sites and/or recording sites in an elongated die;
thinning the die to a thickness below 50 μm; and
applying an etching or dry polishing process for removing sub-surface damage induced by the processing or thinning for increasing the flexibility of the die.

17. The method according to claim 16, further comprising applying an etching process for removing sub-surface damage, wherein the etching process includes applying any of a dry etch or a wet etch.

18. The method according to claim 2, wherein applying a dry etch comprises applying an SF6 based isotropic plasma etch, or wherein applying a wet etch comprises applying an etch in TMAH.

19. A probe device for electrical stimulation and/or recording of activity of excitable cells in a tissue, the probe device comprising:
a plurality of stimulation means and/or recording means located on a die, wherein the die includes a thinned and etched surface for providing flexibility to the probe device.

20. The probe device according to claim 19, wherein the die has a length of at least 30 mm.
